(19) United States
(12) Patent Application Publication
(10) Pub. No.: US 2021/0016070 A1
(43) Pub. Date: Jan. 21, 2021

(54) MULTI-TYPE MICRONEEDLE

(71) Applicant: LABNPEOPLE CO., LTD., Gyeonggi-do (KR)

(72) Inventors: Sung Youn CHO, Gyeonggi-do (KR); Hyun Wook CHOO, Gyeonggi-do (KR); Woo Jin CHUNG, Incheon (KR)

(21) Appl. No.: 17/041,445

(22) PCT Filed: Mar. 29, 2019
(86) PCT No.: PCT/KR2019/003717

§ 371 Date: Sep. 25, 2020

(30) Foreign Application Priority Data
Mar. 30, 2018 (KR) 10-2018-0037037
Mar. 26, 2019 (KR) 10-2019-0034533

(51) Int. Cl. A61M 37/00

(52) U.S. Cl. CPC A61M 37/0015; A61M 2037/0061; A61M 2037/0023

(57) ABSTRACT

A multi-type microneedle is proposed. The multi-type microneedle includes: a substrate part; a plurality of needle holes provided in the substrate part; a plurality of needle parts provided at a predetermined distance from each other along a circumferential direction of each of the needle holes on an area of the substrate part partitioning each of the needle holes; and a patch in contact with a user's skin while in contact with one surface of the substrate part, wherein since the needle parts is provided in a certain pattern around one needle hole by further having a drug injection part or a drug storage part capable of additionally supplying a drug, the needle parts are inserted into a predetermined area of the skin so that the drug to be delivered into the body may be rapidly diffused or permeated subcutaneously, and also an amount of the drug delivered is controlled.

### RELATED APPLICATIONS

The present application is a national phase of International Application Number PCT/KR2019/003717, filed Mar. 29, 2019, which claims priority to Korean Application Number 10-2018-0037037, filed Mar. 30, 2018 and Korean Application Number 10-2019-0034533, filed Mar. 26, 2019.

### TECHNICAL FIELD

The present disclosure relates to a multi-type microneedle and, more particularly, to a microneedle patch that is attached to the skin to deliver a substance such as a drug under the skin.

### BACKGROUND ART

A drug delivery system (DDS) refers to a series of techniques for delivering a substance having pharmacological activity to cells, tissues, organs, etc., by using various physicochemical techniques.

As such a drug delivery system, an oral administration method of ingesting a drug by mouth is most commonly used. In addition, there are other methods such as a transdermal penetration method, in which a drug is delivered to a site of the human body through the skin. Among those methods, a method of delivering a liquid drug by piercing a hole in the skin of a patient with a metal injection needle, that is, a method of delivering a drug by using a syringe, has been widely used for a long time.

However, in the method of delivering the drug by using the syringe, there are disadvantages causing pain to the patient when injecting the drug, causing inconvenience of giving repeated injections, and causing infection in the patient due to reuse of an injection needle resulting from negligence of syringe management.

In addition, the above method of using a syringe also has a disadvantage in that since an inoculator with knowledge of using the syringe is required, a patient is unable to administer the drug by using the syringe by himself or herself.

Therefore, in recent years, in order to improve the drug delivery method using the syringe, a transdermal microneedle having a much smaller size than that of a pen-type syringe has been manufactured and utilized.

The microneedle is a system that delivers a drug by physically piercing small holes in the stratum corneum. In 1998, the Prausnitz Group of Georgia Institute of Technology, USA made a microneedle array with a silicon element by using semiconductor process technology, and proposed applicability of drug delivery. Starting with the above proposal, many studies have been actively conducted, and the microneedle is made in various sizes and shapes on the basis of various materials such as metal, polymer, glass, and ceramic as well as silicon.

In addition, the microneedle is used for the purpose of delivering active substances such as drugs and vaccines in vivo, detecting analytes in the patient's body and performing a biopsy, and injecting other skin beauty substances or drugs into the skin tissue or extracting bodily fluids such as blood from inside the skin. Therefore, since the microneedle may enable a localized and continuous injection with drugs and minimize pain when inserted into the skin, the microneedle may be regarded as one of the drug delivery methods that are rapidly increasing in use in various fields recently.

However, among the conventional microneedles, the microneedle made of polymer materials is not capable of penetrating the stratum corneum of the skin appropriately, and thus has a limitation in physical properties in which a drug is not smoothly diffused into the patient's body. In order to solve this problem, the inventor of the present disclosure has proposed a number of technical details for a microneedle that uses a metal material to increase the rigidity thereof so as to increase power to penetrate into the stratum corneum of the skin, and additionally uses a biodegradable metal material beneficial when remaining in the human body.

That is, as shown in FIG. 1, a microneedle 10 is an initial form of such a microneedle made of the biodegradable metal material, and has the high power to penetrate into the stratum corneum, thereby having increased the drug delivery effect compared to that of the conventional microneedles.

However, as shown in the drawing, since the previously proposed disclosure is configured to include a substrate part 1 attached to the skin and a needle 2 protruding from the substrate part 1 and inserted into the skin, and have a simple structure in which each needle 2 is arranged in a plurality of matrices at a regular distance on the substrate 1, the previously proposed disclosure does not provide an advantageous structure in which a drug to be delivered is accommodated in a microneedle or is passed therethrough, whereby there is a disadvantage of not providing an appropriate means for transdermal delivery of the drug in comparison with the increased penetration efficiency of the needle itself.

Accordingly, in recent years, although a method of reinforcing transdermal drug delivery has been developed steadily by using a chemical enhancer, iontophoresis, electroporation, ultrasound, and thermal elements in order to improve the drug delivery rate, the method has a disadvantage of not only complicating the manufacturing process of the microneedle, but also increasing its manufacturing cost. In addition, there is a case in which application of the method is often unsuitable depending on the type of drugs, and also there is a problem of causing side effects to the skin.

In addition, in general, the substrate part 1 of the microneedle 10 is manufactured by using a mold, and the substrate part 1 manufactured in the mold manner is pressed by a press in a post process. Then, a plurality of needles 2 formed on the substrate 1 may be bent and protrude on the substrate part 1 by being pressed by the press.

However, in the microneedle 10 as shown in FIG. 1, as described above, since the needle 2 has a structure in which a plurality of needles 2 is disposed at a predetermined distance from each other on the substrate part 1, a movable mold having a complex structure, that is, a movable mold provided with a pressing piece respectively corresponding to and in contact with the plurality of needles 2 must be mounted in the press.

In addition, when pressing the plurality of needles 2 with the press equipped with the movable mold as above, there is a problem in that an area of the substrate part 1 disposed between the plurality of needles 2 is affected by the pressing piece and is deformed or damaged.

In particular, in the case of very finely narrowing the distance between the needles 2 by increasing the number of the plurality of needles 2 in order to increase the delivery speed of the drug delivered under the skin, as being disposed closer to the pressing piece, an area of the substrate part 1 disposed between the plurality of needles 2 may be rather easily deformed or damaged.

Therefore, the present applicant has proposed the present disclosure in order to solve the above problems, and as the related art documents, there is “MICRONEEDLE PATCH OF STIMULATING MERIDIAN POINTS” of Korea Patent Application Publication No. 10-2014-0105686, etc.

### DISCLOSURE

### Technical Problem

The present disclosure is to solve the above problems, and an objective of the present disclosure is to provide a multi-type microneedle configured so that a drug to be delivered into a user's body rapidly permeates or diffuses and also an amount of the drug to be delivered into the body is easily adjusted as needed before or after the multi-type microneedle is attached to the skin.

### Technical Solution

The present disclosure includes: a substrate part; a plurality of needle holes provided in the substrate part; a plurality of needle parts provided at a distance from each other along a circumferential direction of each of the needle holes on an area of the substrate part partitioning each of the needle holes; and a patch in contact with a user's skin while in contact with a first surface of the substrate part.

In addition, the patch may be provided with a drug injection part into which a drug to be delivered into a body is injected.

In addition, the drug injection part may be provided on the patch in a form of an opening connected in communication with the plurality of needle holes provided in the substrate part.

In addition, at least one or more of the drug injection part may be provided on the substrate part.

In addition, the substrate part may be provided with a drug storage part in which a drug to be delivered into the body is stored.

In addition, the drug storage part may be interposed between the substrate part and the patch in a form of an absorbent pad.

In addition, the drug storage part may be made of a non-absorbent material, be interposed between the substrate part and the patch, and have an annular ring shape.

In addition, the drug storage part may have a shape protruding outward on the patch while forming a predetermined space part in which the drug is able to be stored.

In addition, the drug storage part may include: a storage member provided to protrude to an outside of the patch while forming the space part and provided with a first side having an opening so as to allow the drug to flow; and a connection member integrally connected to a circumferential surface of the first side of the storage member and interposed between the substrate part and the patch.

In addition, the multi-type microneedle may further include a blocking member blocking the open first side of the storage member.

In addition, the storage member may protrude on the patch in a shape having a hemispherical cross section or a shape having a “C”-shaped cross section.

In addition, the opening formed at the first side of the storage member may have an area including all of the plurality of needle holes provided in the substrate part, or an area including some of the plurality of needle holes provided in the substrate part.

In addition, a plurality of drug storage parts may be provided on the substrate part.

In addition, each of the needle holes may be provided in the substrate part in a circular or regular polygonal shape.

In addition, each of the needle parts may be provided in a center of an inner side of the substrate part partitioning each of the needle holes when each of the needle holes is formed in the regular polygonal shape.

In addition, a first end of each of the needle parts may be connected to the area of the substrate part partitioning each of the needle holes, and a second end of each of the needle parts may protrude from a second surface of the substrate part and be inserted into the user's skin.

In addition, the multi-type microneedle may further include an accommodating groove provided on each of the needle parts or the substrate part so as to provide a path through which a drug is able to flow.

In addition, the accommodating groove may be provided on the first surface or the second surface of the substrate part, a first end in a longitudinal direction of the accommodating groove may be connected to a part of the area formed by the substrate part, and a second end in the longitudinal direction of the accommodating groove may extend toward the second end of each of the needle parts.

In addition, the multi-type microneedle may further include a slit groove connected in communication from the second end in the longitudinal direction of the accommodating groove to a tip of each of the needle parts.

In addition, an accommodating hole may be formed along a direction in which the accommodating groove is formed in the area of the substrate part or each of the needle parts where the accommodating groove is formed.

In addition, the substrate part may have a honeycomb structure when each of the needle holes is formed in a regular hexagonal shape.

In addition, the substrate part or each of the needle parts may be formed of a bioabsorbable metal.

In addition, the bioabsorbable metal may be a metal containing at least one component of magnesium, calcium, zinc, and iron.

### Advantageous Effects

Since the multi-type microneedle according to an exemplary embodiment of the present disclosure has a configuration in which a plurality of needle parts is provided in a predetermined pattern around one needle hole, the plurality of needle parts is inserted into a predetermined area of the skin, whereby the drug to be delivered to the skin may quickly diffuse or permeate subcutaneously.

In addition, since the multi-type microneedle according to the exemplary embodiment of the present disclosure has a structure capable of controlling the amount of drug delivered into the body through a drug injection part or a drug storage part, the multi-type microneedle may be used in correspondence with the user's preference or the patient's condition.

In addition, since the multi-type microneedle according to the exemplary embodiment of the present disclosure has a structure capable of controlling the amount of drug delivered into the body before or after the multi-type microneedle is attached to the skin, the multi-type microneedle may be easily used in correspondence with the physical condition of the user.

In addition, since the multi-type microneedle according to the exemplary embodiment of the present disclosure is provided with a substrate part that may have a honeycomb structure by the needle holes formed in the shape of a regular hexagon, it is possible to minimize deformation or damage of the substrate part by the press in the press pressurization process of bending the needle part.

In addition, since the multi-type microneedles according to the exemplary embodiment of the present disclosure may allow the plurality of needle parts to protrude from the substrate part without having to provide the same number of pressing pieces, provided in the movable mold of the press, as the number of needle parts, the manufacturing cost of the movable mold of the press is reduced, whereby the manufacturing cost of the microneedle may be reduced consequently and the manufacturing process may be simplified as well.

### DESCRIPTION OF DRAWINGS

FIG. 1 is a perspective view of a conventional microneedle.

FIG. 2 is a perspective view of a multi-type microneedle according to an exemplary embodiment of the present disclosure.

FIG. 3 is an exploded perspective view of the multi-type microneedle shown in FIG. 2.

FIG. 4 is a plan view of the multi-type microneedle shown in FIG. 2.

FIG. 5 is a rear view of the multi-type microneedle shown in FIG. 2.

FIG. 6 is a perspective view of the multi-type microneedle provided with a drug storage part according to the exemplary embodiment of the present disclosure.

FIG. 7 is an exploded perspective view of the multi-type microneedle shown in FIG. 6.

FIG. 8 is a perspective view of a multi-type microneedle provided with a drug storage part according to another exemplary embodiment of the present disclosure.

FIG. 9 is an exploded perspective view of the multi-type microneedle shown in FIG. 8.

FIG. 10 is a perspective view of a multi-type microneedle provided with a drug storage part according to still another exemplary embodiment of the present disclosure.

FIG. 11 is a cross-sectional view of the drug storage part shown in FIG. 10.

FIG. 12 is a perspective view of the drug storage part configured as a storage member having a cross-section of a “C” shape.

FIG. 13A is a perspective view showing a state in which the drug storage part shown in FIG. 8 is provided on a different type of substrate part.

FIG. 13B is a cross-sectional view of the multi-type microneedle shown in FIG. 13A.

FIG. 14A is a perspective view showing a state in which the drug storage part shown in FIG. 10 is provided on a different type of substrate part.

FIG. 14B is a cross-sectional view of the multi-type microneedle shown in FIG. 14A.

### MODE FOR INVENTION

Advantages and features of the present disclosure, and a method of achieving them will become apparent with reference to the exemplary embodiments described below in detail together with the accompanying drawings.

However, the present disclosure is not limited to the exemplary embodiments disclosed below, but will be implemented in a variety of different forms. These exemplary embodiments are provided only to complete the disclosure of the present disclosure and to completely inform the scope of the present disclosure to those skilled in the art to which the present disclosure pertains, and the present disclosure is only defined by the scope of the claims.

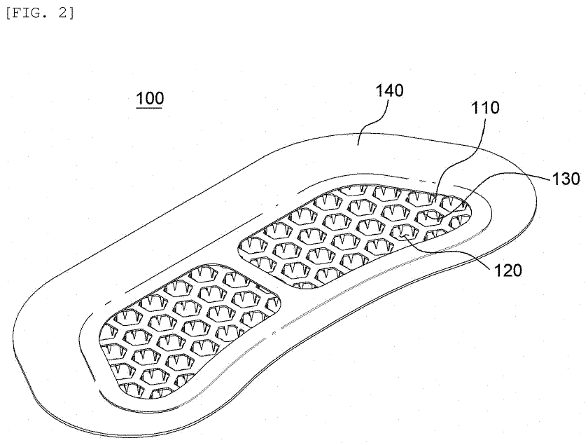

Hereinafter, a multi-type microneedle according to an exemplary embodiment of the present disclosure will be described in detail with reference to FIGS. 2 to 12. In describing the present disclosure, detailed descriptions of related known functions or configurations are omitted so as not to obscure the subject matter of the present disclosure.

As shown in FIGS. 2 to 12, the multi-type microneedle 100 according to the exemplary embodiment of the present disclosure may include: a substrate part 110; a plurality of needle holes 120 provided in the substrate part 110; a plurality of needle parts 130 provided at a predetermined distance from each other along a circumferential direction of each of the needle holes 120 on an area of the substrate part 110 partitioning each of the needle holes 120; and a patch 140 in contact with a user's skin while in contact with a first surface of the substrate part 110.

The substrate part 110 may have a form of a thin metal plate having a predetermined area and thickness, the first surface of the substrate part is in contact with the patch 140, and a second surface of the substrate part is in contact with the user's skin.

In addition, the substrate part 110 may be manufactured in various sizes and shapes corresponding to a site of the skin where the substrate part is to be attached, and may be provided with a circumferential part thereof formed to have various curvatures so as to be in airtight contact with a curved skin site. For example, when attached to a user's nose, the substrate part 110 may also be manufactured in the form such as a known nose pack.

In addition, a drug to be delivered under the skin may be accommodated in the substrate part 110. As a method of accommodating the drug in the substrate part 110, various known methods may be used, including a method of coating by immersing the substrate part 110 in a container in which the drug is stored, or a method of coating by applying the drug to the substrate part 110.

For reference, the drug accommodated in the substrate part 110 may be a drug for the purpose of preventing and treating diseases, but is not limited thereto, and the drug may be a genetic material, EGF (Epidermal Growth Factor) for skin beauty, or hyaluronic acid.

The needle hole 120 is a component that may be formed by processing the substrate part 110 by a known laser cutting device or etching molding method, and as described above, the plurality of needle holes may be provided on the surface of the substrate part 110 at a predetermined distance from each other.

In addition, the needle hole 120 may be formed in the substrate part 110 in a circular or regular polygonal shape.

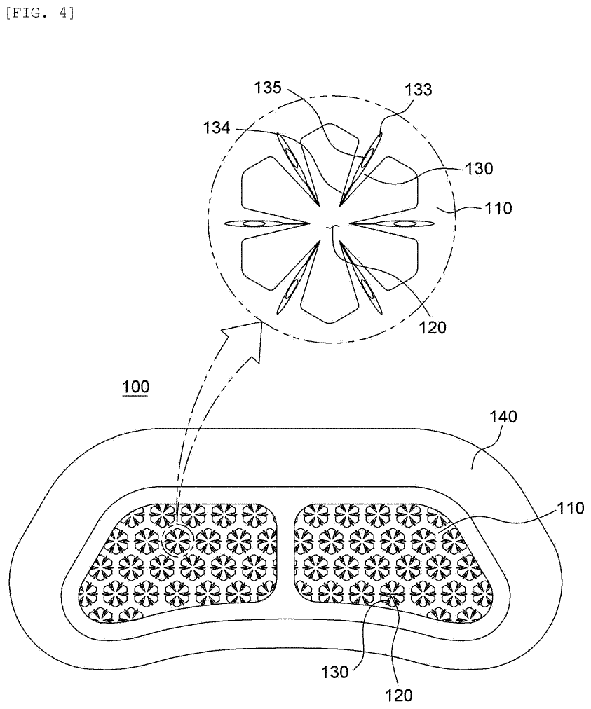

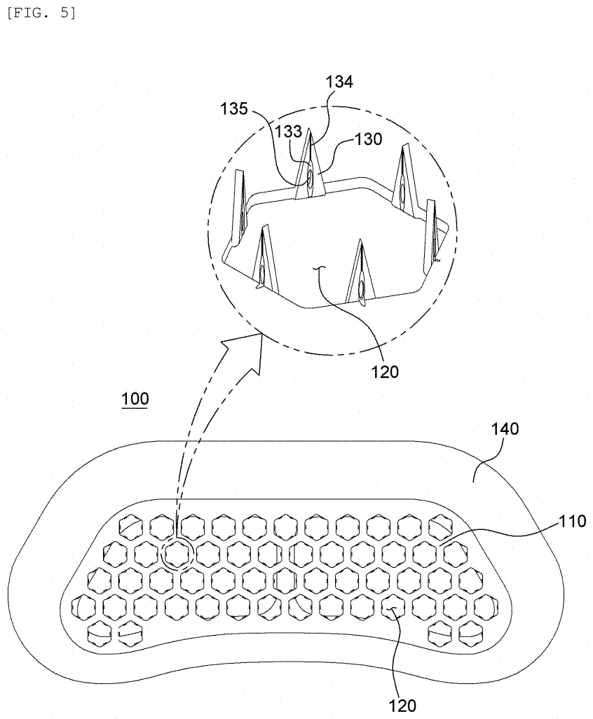

As shown in FIGS. 4 and 5, the needle part 130 may be provided on an inner surface of the substrate part 110 partitioning each of the needle holes 120. At this time, when the needle hole 120 is formed in a regular polygonal shape, the needle part 130 may be provided in the center of the inner side of the substrate part 110 partitioning each of the needle holes 120.

That is, a first end in the longitudinal direction of each of the needle parts 130 is connected to an area of the substrate part 110 partitioning each of the needle holes 120, and a second end in the longitudinal direction thereof may be extended toward the center of the needle hole 120.

Here, as shown in FIG. 4, the needle part 130 may be bent to protrude in a vertical direction on the second surface of the substrate part 110 by a molding process or a pressing process using a press device. That is, the needle part 130 may be referred to as a component in which a drug accommodated in the substrate part 110 and the needle part itself is delivered by the needle part inserted in the skin of the user subcutaneously when the substrate part 110 comes into contact with the user's skin, and may be referred to as a component in which a drug injected into a drug injection part 150 to be described later is also delivered under the skin of the user.

Meanwhile, an accommodating groove 133 may be further provided in the needle part 130 and the substrate part 110.

The accommodating groove 133 is provided on the first surface or the second surface of the substrate part 110, and the first end in the longitudinal direction of the accommodating groove is disposed in a part of an area formed by the substrate part 110, and the second end in the longitudinal direction of the accommodating groove may be disposed extending toward the tip of each of the needle parts 130, that is, the second end in the longitudinal direction of each of the needle parts 130.

The accommodating groove 133 may be selectively provided on a first side or the other side of the substrate part 110, and in the exemplary embodiment of the present disclosure, it is shown in the drawings that the accommodating groove is provided on both the first side and the other side of the substrate part 110.

The accommodating groove 133 as described above not only forms a path through which the drug accommodated in the substrate part 110 or the needle part 130 may flow, but also provides a space in which the drug accommodated in the substrate part 110 or the needle part 130 may be stored, so that the amount of drug delivered into the body may be controlled.

In addition, the accommodating groove 133 may have a shape in which the inner diameter thereof is tapered toward the thickness direction of the substrate part 110 or the needle part 130.

The accommodating groove 133 configured as described above not only allows the amount of the drug accommodated in the substrate part 110 or the needle part 130 to be adjusted by increasing the plane area of the substrate part 110 or the needle part 130 according to the shape, but also may provide a flow path so that the drug accommodated in the substrate part 110 or the needle part 130 may be easily delivered under the skin.

In other words, in a case where the needle part 130 composed only of a flat surface without a structure such as the accommodating groove 133 in accordance with the drug delivery path is inserted into the skin, the case forms a structure in which it is difficult for the drug to flow into the skin due to the fact that the skin and the plate surface of each of the needle parts 130 are in close contact with each other, whereas, when the needle part 130 is inserted into the skin as the substrate part 110 and the needle part 130 on which the accommodating groove 133 is formed come in contact with the user's skin, the drug stored in the accommodating groove 133 may be easily delivered and diffused into the body. In addition, the drug accommodated in the substrate part 110 or the needle part 130 may flow along a formation direction of the accommodating groove 133 and be delivered under the skin.

In addition, the multi-type microneedle 100 according to the exemplary embodiment of the present disclosure may further include a slit groove 134 connecting the second end in the longitudinal direction of the accommodating groove 133 to the tip of each of the needle parts 130, that is, the second end in longitudinal direction of each of the needle parts 130 so as to communicate with each other.

The slit groove 134 serves to allow the drug stored in the accommodating groove 133 or an accommodating hole 135 to be described later to easily flow to the tip of each of the needle parts 130, and accordingly, the drug contained in the substrate part 110 or the needle part 130 may be more easily delivered under the skin of the user through the accommodating groove 133 and the slit groove 134 in sequence.

Meanwhile, the accommodating hole 135 may be further provided in the accommodating groove 133. That is, the accommodating hole 135 may be formed along the direction in which the accommodating groove 133 is formed in the area of the substrate part 110 or the needle part 130 where the accommodating groove 133 is formed.

Since the accommodating hole 135 connects the first surface and the second surface of the substrate part 110 or the first surface and the second surface of each of the needle parts 130 to each other to communicate with each other, a drug accommodated in the needle part 130, or the first surface or the second surface of the substrate part 110 may communicate with each other, so that the drug may diffuse rapidly in the user's skin.

In addition, since the accommodating hole 135 provides a space in which the drug may be stored as the same as the accommodating groove 133, the amount of the drug accommodated in the substrate part 110 or the needle part 130 may be adjusted. That is, since the accommodating hole 135 may be provided in a form in which the inner diameter is tapered from the first surface of each of the needle parts 130 or the substrate part 110 toward the second surface direction thereof, or is tapered from the second surface of each of the needle parts 130 or the substrate part 110 toward the first surface direction thereof, a space to receive a drug coating layer formed by being accommodated in the needle part 130 may be provided.

In the multi-type microneedle 100 according to the exemplary embodiment of the present disclosure configured as described above, since the plurality of needle parts 130 is provided at a predetermined distance on the needle hole 120 formed on the substrate part 110, a drug to be supplied into the body may be intensively delivered under the skin and may be diffused quickly.

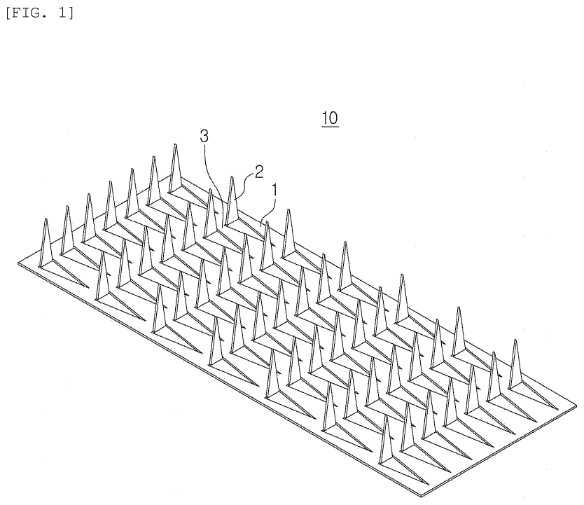

In addition, when the needle hole 120 is formed in a regular hexagonal shape, the substrate part 110 may have a honeycomb structure. In the substrate part 110 having the honeycomb structure, an area 110a (refer to FIG. 1) of the substrate part 110 partitioning each of the needle holes 120 may be prevented from being deformed or damaged by a pressing piece in the process of pressing with the pressing piece of a press in order to bend the plurality of needle parts 130 disposed around the needle hole 120.

To be more specific, in the process where the pressing piece provided in the movable mold of the press is inserted into the needle hole 120, the needle part 130 is pressed and bent, but at this time, there is concern that the area 110a (refer to FIG. 1) of the substrate part 110 disposed between the plurality of needle holes 120 may be affected by the pressing piece 110 and is deformed or torn.

However, the multi-type microneedle 100 according to the exemplary embodiment of the present disclosure may allow the substrate part 110 to be formed in a honeycomb structure by providing the needle hole 120 in the form of a regular hexagon, and accordingly, by increasing the overall rigidity of the substrate part 110, it is possible to prevent the substrate part 110 from being deformed or damaged in the work process using the press.

In addition, the plurality of needle parts 130 is pressed and bent by one pressing piece inserted into one needle hole 120 to protrude from the substrate part 110. Therefore, it is not necessary to provide the number of pressing pieces provided in the movable mold of the press to a number corresponding to the number of each of the needle parts 130, so that the structure of the movable mold may be simplified as a whole. Accordingly, as the manufacturing cost of the mold is reduced, the manufacturing cost of the multi-type microneedle 100 may be reduced, and the manufacturing process thereof may be simplified, as a result.

Meanwhile, the multi-type microneedle 100 according to the exemplary embodiment of the present disclosure may be formed of a bioabsorbable metal. That is, the substrate part 110 or the needle part 130 may be formed of the bioabsorbable metal composed of a component beneficial to the human body.

That is, the substrate part 110 may be made of a metal containing at least one component of magnesium, calcium, zinc, and iron used as a bioabsorbable metal, and accordingly, the needle part 130 provided on the substrate part 110 is also made of the bioabsorbable metal.

For reference, as for the bioabsorbable metal, there is a case where an alloy based on magnesium is produced and commercialized at home and abroad for application as an orthopedic implant, and the bioabsorbable metal applied to the orthopedic implant has focused on reducing the decomposition rate as much as possible inside the body or improving the corrosion resistance for safe fracture fixation.

However, unlike the bioabsorbable metal applied to orthopedics, the bioabsorbable metal forming the multi-type microneedle 100 according to the exemplary embodiment of the present disclosure accelerates the decomposition rate in the body, and thus a mechanism capable of supplying minerals may be applied together with administering the drug under the skin.

For example, magnesium, calcium, and zinc used as bioabsorbable metals have a mechanism of reacting with water to release hydrogen gas and be decomposed, as shown in the following [Chemical Formula 1] to [Chemical Formula 3].

Mg+2H₂O→Mg(OH)₂+H₂(gas) [Chemical Formula 1]

Ca+2H₂O→Ca(OH)₂+H₂(gas) [Chemical Formula 2]

Zn+2H₂O→Zn(OH)₂+H₂(gas) [Chemical Formula 3]

The substrate part 110 and the needle part 130 formed of the bioabsorbable metal as described above release ions and decomposition products under the skin, and hydrogen gas generated by by-product provides a swelling effect under the skin, thereby inducing a wrinkle improvement effect.

In addition, while remaining on the skin surface under the skin, ZnO and MgCl which are the by-products generated by inserting magnesium and zinc being constituents of bioabsorbable metals in vivo may also serve as a drug delivery enhancer that improves absorbing subcutaneously the drug accommodated in the substrate part 110 and the needle part 130. Accordingly, the substrate part 110 and the needle part 130 formed of the bioabsorbable metal may effectively deliver the drug accommodated in itself to a user.

Meanwhile, a shape of the needle hole 120 and the needle part 130 formed in the substrate part 110 may be provided by patterning the shape on the substrate part 110 by a known lithography or etching technique.

At this time, since the substrate part 110 formed of a bioabsorbable metal has a lower corrosion resistance than that of a metal made of a metallic material such as stainless steel or iron, the edge of each of the needle parts 130 is etched by a technique of lithography or etching so as to be thinner than the thickness of the raw material itself, thereby having a sharp shape that may be easily inserted in the skin subcutaneously.

In addition, since the multi-type microneedle 100 according to the exemplary embodiment of the present disclosure is provided with a needle hole 120 having a relatively larger diameter or a larger area than that of the needle hole formed in the conventional microneedle, the user may also be able to receive light therapy through the needle hole 120 simultaneously.

That is, even though the substrate part 110 is attached to the skin, since the user's skin may be exposed to the outside through the plurality of needle holes 120, light therapy may also be easily performed by illuminating the exposed skin with light beneficial to cure skin pain and skin diseases such as acne, and atopy.

Therefore, the user may receive treatment by the drug injected into the skin and diffused therein through the needle part 130 and by the light emitted through the needle hole 120, so that the synergistic effect of drug treatment and light therapy may be expected.

For reference, in the exemplary embodiment of the present disclosure, it has been described that the user may also receive the light therapy by illuminating the exposed skin with the light, which is beneficial to the human body, through the needle hole 120, but the effect is not limited thereto. That is, a skin treatment procedure may also be performed by applying a liquid skin care material or a skin treatment material to the exposed skin through the needle hole 120.

The patch 140 is a component for enabling the second surface of the substrate part 110 to closely contact with the user's skin, so that the patch may be attached to the first surface of the substrate part 110 as described above.

While having an area larger than the area formed by the substrate part 110, the patch 140 may be attached to the first surface of the substrate part 110, and on the surface facing the first surface of the substrate part 110, an adhesive material is coated, or a material having good adhesion to the skin is provided.

The area of the patch 140 that is not in contact with the substrate part 110 may be referred to as an area where the patch is attached to the user's skin, and accordingly the substrate part 110 is prevented from moving freely on the user's skin.

In addition, the patch 140 may be equipped with a drug injection part 150 that provides a path through which a drug may be injected to the first side of the substrate part 110 while the second surface of the substrate part 110 is in contact with the user's skin.

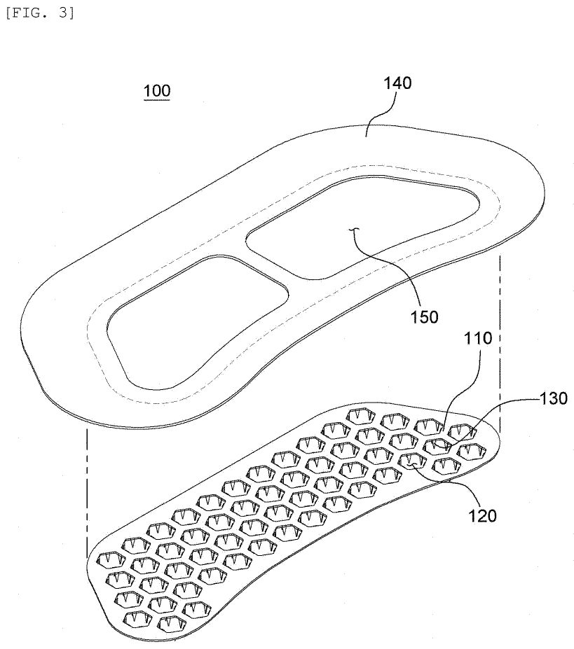

As shown in FIGS. 2 and 3, the drug injection part 150 may be provided on the patch 140 in the form of an opening connected in communication with the plurality of needle holes 120 provided in the substrate part 110, and at least one or more of the drug injection parts may be provided on the patch 140. In the exemplary embodiment of the present disclosure, it is shown in the drawings that two openings are formed in the patch 140 at a predetermined distance from each other.

Accordingly, the user may be able to additionally apply a drug on the substrate part 110 through the drug injection part 150, and the drug applied on the substrate part 110 may be delivered to the skin of the user via the needle hole 120 and the needle part 130.

For reference, a method of injecting a drug into the drug injection part 150 may be performed through a drug feeder having the form of a syringe, and may also be performed through an ampoule container in which the drug is stored. In addition, a method of attaching a drug-impregnated sheet may be applied.

That is, in addition to drugs primarily accommodated on the substrate part 110, the user may use drugs by additionally supplementing the substrate part 110 with the drug stored in the ampoule container or the syringe, and the drug impregnated in the sheet, etc.

Accordingly, even though the drug primarily accommodated in the substrate part 110 is completely consumed by drying out or by being administered subcutaneously, since the substrate part 110 may be maintained without being removed from the skin, the multi-type microneedle 100 of the present disclosure has the advantage of being usable for a longer time.

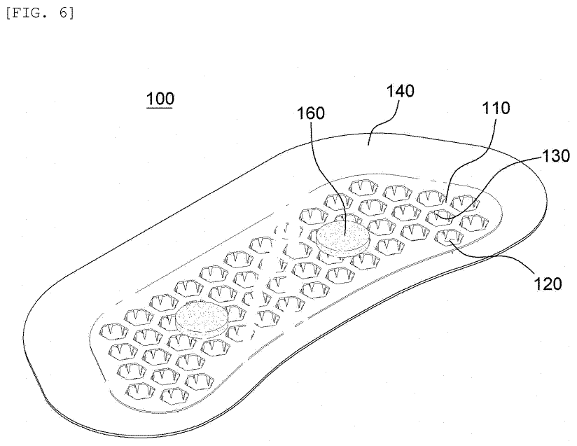

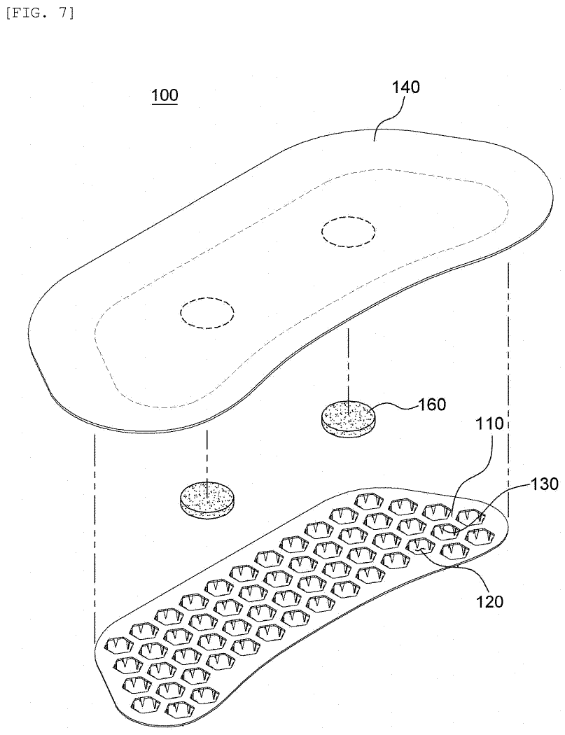

Meanwhile, as shown in FIGS. 6 and 7, a drug storage part 160 for storing a drug to be delivered to the user's body may be further provided on the substrate part 110.

The drug storage part 160 may be interposed between the first surface of the substrate part 110 and the patch 140 in the form of an absorbent pad.

The absorbent pad may be made of a material such as a known sponge, cotton, and polyurethane that may hold a liquid material for a long time. For reference, in the exemplary embodiment of the present disclosure, it is shown in the drawings that two absorbent pads having a circular shape are provided between the first surface of the substrate part 110 and the patch 140 at a predetermined distance from each other, but the present disclosure is not limited thereto, and at least one or more of the pads may be provided on the substrate part 110.

The drug storage part 160 as described above may be referred to as a component that enables a user to additionally supplement a drug through the second surface of the substrate part 110 before attaching the substrate part 110 to the user's skin. That is, the user may additionally supply the drug to the second surface of the substrate part 110 by using the above-described syringe or ampoule container, and at this time, the supplied drug may be absorbed by the drug storage part 160 interposed between the first surface of the substrate part 110 and the patch 140.

Accordingly, the user may be able to increase or decrease the amount of drug to suit his or her preference before attaching the substrate part 110 to the skin, and in the case where the multi-type microneedle 100 is used on a patient for medical purposes, a doctor or nurse may adjust the amount of the drug depending on the patient's condition, through the drug storage part 160.

For reference, when the drug storage part 160 is provided on the substrate part 110, it is preferable that the drug injection part 150 is not formed in the patch 140. This is because the drug absorbed in the drug storage part 160 may be easily evaporated by wind or air flowing through the drug injection part 150.

In addition, the drug storage part 160 having the form of an absorbent pad may have a size or area capable of including all of the plurality of needle holes 120 provided in the substrate part 110, or may have a size or area capable of including some of the plurality of needle holes 120. For reference, in FIGS. 6 and 7, it is shown in the diagrams that the drug storage part 160 having an absorbent pad shape is provided on the substrate part 110 while having a size or area capable of including some of the needle holes 120 among the plurality of needle holes 120 formed in the substrate part 110.

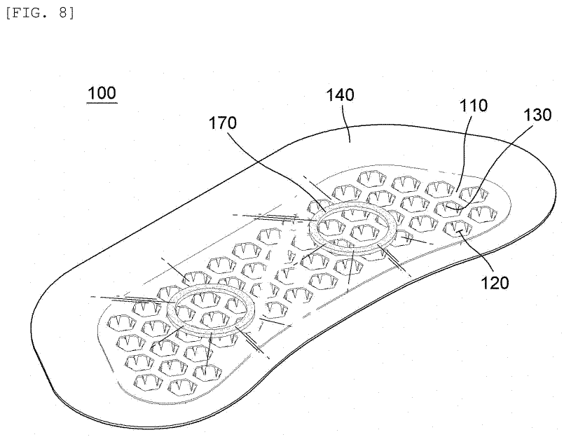

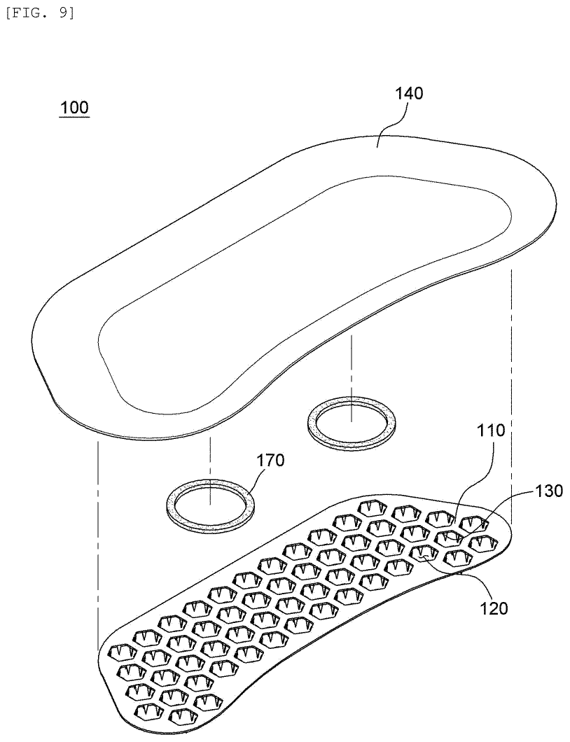

Hereinafter, a drug storage part 170 according to another exemplary embodiment of the present disclosure will be described with reference to FIGS. 8 and 9.

As shown in FIGS. 8 and 9, the drug storage part 170 according to another exemplary embodiment of the present disclosure is made of a non-absorbent material and is interposed between a first surface of a substrate part 110 and a patch, and may have an annular ring shape.

The non-absorbent material may be made of a thermoplastic plastic material such as polyethylene. For reference, in the exemplary embodiment of the present disclosure, it is shown in the drawings that two non-absorbent materials having the annular ring shape are provided between the first surface of the substrate 110 and the patch 140 at a predetermined distance from each other.

As described above, a drug storage part 170 made of the non-absorbent material allows a drug to be delivered intensively to a user's skin area by partitioning a space in which the drug supplemented by a user may be stored, rather than absorbing and storing the drug, and also serves to prevent the drug injected from a syringe or ampoule container from soaking into the patch 140 by leaking out of the area formed by the substrate part 110.

In other words, since the drug storage part 170 made of the non-absorbent material is provided on the substrate part 110 while having a ring shape, when the user additionally supplies the drug to an inside of the drug storage part 170 by using the syringe or ampoule container, the supplied drug may be stored in an internal space partitioned by the drug storage part 170.

For reference, when the drug storage part 170 made of the non-absorbent material is provided on the substrate part 110, it is preferable that a drug injection part 150 is not formed in the patch 140. This is because the drug absorbed in the drug storage part 170 may be easily evaporated by wind or air flowing through the drug injection part 150.

In addition, the drug storage part 170 in a ring shape made of the non-absorbent material may have a size or area that may include all of a plurality of needle holes 120 provided in the substrate part 110, or may have a size or area that may include some of the plurality of needle holes 120. For reference, in FIGS. 8 and 9, it is shown in the drawings that the drug storage part 170 having a ring shape made of a non-absorbent material is provided on the substrate part 110 while having the size or area capable of including some of the needle holes 120 among the plurality of needle holes 120 formed in the substrate part 110.

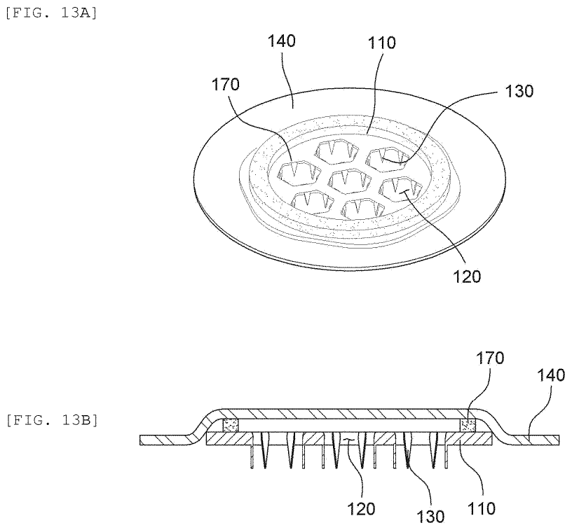

For reference, in FIGS. 13A and 13B, it is shown in the drawing that the drug storage part 170 in a ring shape made of the non-absorbent material is provided on the substrate part 110 while having the size or area capable of including all of the plurality of needle holes 120 provided in the substrate part 110. That is, the drug storage part 170 may be provided on the substrate part 110 having a substantially regular hexagonal shape, and all of the plurality of needle holes 120 provided in the substrate part 110 may be disposed in the space partitioned by the drug storage part 170.

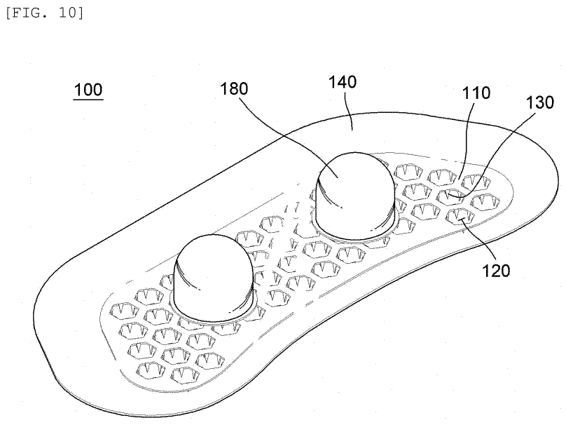

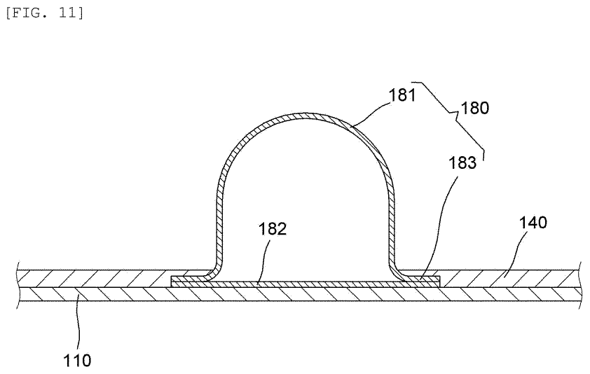

Hereinafter, a drug storage part 180 according to still another exemplary embodiment of the present disclosure will be described with reference to FIGS. 10 and 11.

As shown in FIGS. 10 and 11, a drug storage part 180 according to still another exemplary embodiment of the present disclosure may have a shape protruding outward on a patch 140 while forming a predetermined space part in which a drug may be stored. That is, the drug storage part 180 according to still another exemplary embodiment of the present disclosure may be provided on a substrate part 110 while having a shape that may be pressed by a user's hand.

The drug storage part 180 is provided to protrude to the outside of the patch 140 while forming the space part, and may include: a storage member 181 provided with a first side thereof having an open shape to allow the drug to flow; and a connection member 183 integrally connected to a circumferential surface of the first side of the storage member 181 and interposed between the substrate part 110 and the patch 140.

The storage member 181 provides a space in which the drug is stored and may be referred to as a component pressed by the user's hand. As shown in FIGS. 10 and 11, such a storage member 181 may be provided on the substrate part 110 in a shape having a hemispherical cross section and protrude to the outside of the patch 140.

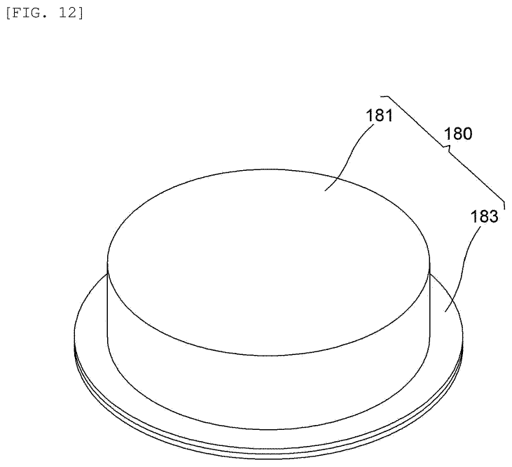

As long as forming a space in which a drug may be stored and may be easily pressed by the user's hand at the same time, the storage member 181 is obviously not limited to the above-described hemispherical shape, and may have various shapes. For example, as shown in FIG. 12, the storage member 181 may be provided on the substrate part 110 in the form having a “C”-shaped cross section.

Here, in an area of the patch 140 corresponding to the storage member 181, a through hole may be formed so that the storage member 181 protrudes to the outside of the patch 140 to be pressed by the user's hand.

The connection member 183 may be referred to as a component joined to a first surface of the substrate part 110, and may be interposed between the substrate part 110 and the patch 140 as described above.

As described above, a drug storage part 180 according to still another exemplary embodiment of the present disclosure composed of the storage member 181 and the connection member 183 may be made of a transparent thermoplastic resin or film material.

The drug storage part 180 as described above enables a user to additionally supplement a drug through a second surface of the substrate part 110 before the user attaches the substrate part 110 to the user's skin. That is, the user may additionally supply the drug to the space part formed by the storage member 181 through the second surface of the substrate part 110 by using the above-described syringe or ampoule container.

Therefore, the user may be able to increase or decrease the amount of drug so as to suit his or her preference before attaching the substrate part 110 to the user's skin, and also when the multi-type microneedle 100 is used on a patient for medical purposes, a doctor or a nurse may adjust the supply amount of the drug depending on the patient's condition through the drug storage part 180.

Meanwhile, as shown in FIG. 11, the open first side of the storage member 181 may be blocked by a blocking member 182, so that the drug stored in the storage member 181 may leak from the storage member 181 only when being pressed by the user's hand.

That is, the open first side of the storage member 181 may be blocked by the blocking member 182, so that the drug stored in the storage member 181 may be selectively used by the user. Accordingly, when the user presses the storage member 181 by hand, the blocking member 182 is torn by the pressure, so that the drug stored in the storage member 181 may flow out onto the substrate part 110.

Therefore, the drug storage part 180 according to still another exemplary embodiment of the present disclosure may be used in a way where the user supplements the drug in the storage member 181 by using a syringe or ampoule container. Alternatively, the drug storage part 180 may be used in a way of being provided on the substrate part 110 in a packaged state in which the drug is already stored.

In addition, the opening formed on a first side of the drug storage part 180 may have a size or area capable of including all of the plurality of needle holes 120 provided in the substrate part 110, or may have a size or area capable of including some of the plurality of needle holes 120. For reference, in FIGS. 10 and 11, it is shown in the drawings that the drug storage part 180 is provided on the substrate part 110, while having the size or area capable of including some of the needle holes 120 among the plurality of needle holes 120 formed in the substrate part 110

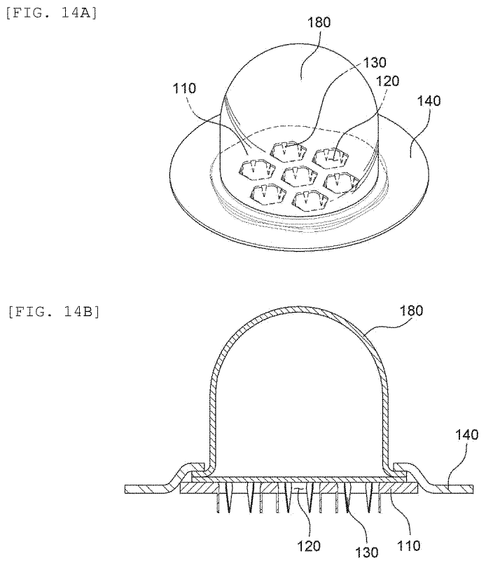

For reference, in FIGS. 14A and 14B, it is shown in the drawing that the opening of the drug storage part 180 provided to protrude outward of the patch 140 may be provided in the substrate part 110 while having the size or area capable of including all of the plurality of needle holes 120 provided in the substrate part 110. That is, the drug storage part 180 may be provided on the substrate part 110 having a substantially regular hexagonal shape, and all of the plurality of needle holes 120 provided in the substrate part 110 may be disposed in the opening on the first side formed by the drug storage part 180.

Although detailed exemplary embodiments according to the present disclosure have been described so far, obviously, various modifications may be made without departing from the scope of the present disclosure.

Therefore, the scope of the present disclosure should not be limited to the described exemplary embodiments, and should be determined not only by the scope of the claims to be described later, but also by the scope and equivalents of the claims.

### INDUSTRIAL APPLICABILITY

The multi-type microneedle of the present disclosure may be sold and used in various industries such as medical fields and skin care fields.

1. A multi-type microneedle comprising:
a substrate part;
a plurality of needle holes provided in the substrate part;
a plurality of needle parts provided at a distance from each other along a circumferential direction of each of the needle holes on an area of the substrate part partitioning each of the needle holes; and
a patch in contact with a user's skin while in contact with a first surface of the substrate part.

2. The multi-type microneedle of claim 1, wherein the patch is provided with a drug injection part into which a drug to be delivered into a body is injected.

3. The multi-type microneedle of claim 2, wherein the drug injection part is provided on the patch in a form of an opening connected in communication with the plurality of needle holes provided in the substrate part.

4. The multi-type microneedle of claim 3, wherein at least one or more of the drug injection part is provided on the substrate part.

5. The multi-type microneedle of claim 1, wherein the substrate part is provided with a drug storage part in which a drug to be delivered into the body is stored.

6. The multi-type microneedle of claim 5, wherein the drug storage part is interposed between the substrate part and the patch in a form of an absorbent pad.

7. The multi-type microneedle of claim 5, wherein the drug storage part is made of a non-absorbent material, is interposed between the substrate part and the patch, and has an annular ring shape.

8. The multi-type microneedle of claim 5, wherein the drug storage part has a shape protruding outward on the patch while forming a predetermined space part in which the drug is able to be stored.

9. The multi-type microneedle of claim 8, wherein the drug storage part comprises:
a storage member provided to protrude to an outside of the patch while forming the space part and provided with a first side having an opening so as to allow the drug to flow; and
a connection member integrally connected to a circumferential surface of the first side of the storage member and interposed between the substrate part and the patch.

10. The multi-type microneedle of claim 9, further comprising:
a blocking member blocking the open first side of the storage member.

11. The multi-type microneedle of claim 9, wherein the storage member protrudes on the patch in a shape having a hemispherical cross section or a shape having a “C”-shaped cross section.

12. The multi-type microneedle of claim 9, wherein the opening formed at the first side of the storage member has an area including all of the plurality of needle holes provided in the substrate part, or an area including some of the plurality of needle holes provided in the substrate part.

13. The multi-type microneedle of claim 6, wherein a plurality of drug storage parts is provided on the substrate part.

14. The multi-type microneedle of claim 1, wherein each of the needle holes is provided in the substrate part in a circular or regular polygonal shape.

15. The multi-type microneedle of claim 14, wherein each of the needle parts is provided in a center of an inner side of the substrate part partitioning each of the needle holes when each of the needle holes is formed in the regular polygonal shape.

16. The multi-type microneedle of claim 1, wherein
a first end of each of the needle parts is connected to the area of the substrate part partitioning each of the needle holes, and
a second end of each of the needle parts protrudes from a second surface of the substrate part and is inserted into the user's skin.

17. The multi-type microneedle of claim 16, further comprising:
an accommodating groove provided on each of the needle parts or the substrate part so as to provide a path through which a drug is able to flow.

18. The multi-type microneedle of claim 17, wherein
the accommodating groove is provided on the first surface or the second surface of the substrate part,
a first end in a longitudinal direction of the accommodating groove is connected to a part of the area formed by the substrate part, and
a second end in the longitudinal direction of the accommodating groove extends toward the second end of each of the needle parts.

19. The multi-type microneedle of claim 18, further comprising:
a slit groove connected in communication from the second end in the longitudinal direction of the accommodating groove to a tip of each of the needle parts.

20. The multi-type microneedle of claim 18, wherein an accommodating hole is formed along a direction in which the accommodating groove is formed in the area of the substrate part or each of the needle parts where the accommodating groove is formed.

21. The multi-type microneedle of claim 14, wherein the substrate part has a honeycomb structure when each of the needle holes is formed in a regular hexagonal shape.

22. The multi-type microneedle of claim 1, wherein the substrate part or each of the needle parts is formed of a bioabsorbable metal.

23. The multi-type microneedle of claim 22, wherein the bioabsorbable metal is a metal containing at least one component of magnesium, calcium, zinc, and iron.
